(19) United States
(12) Patent Application Publication
(10) Pub. No.: US 2005/0081258 A1
(43) Pub. Date: Apr. 14, 2005

(54) Short fragment homologous recombination to effect targeted genetic alterations in plants

(76) Inventor: Nicole Lesley Prokopishyn, Levittown, PA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/501,856

(22) PCT Filed: Jan. 21, 2003
(86) PCT No.: PCT/US03/01734

Related U.S. Application Data
(60) Provisional application No. 60/350,161, filed on Jan. 18, 2002.

(51) Int. Cl. A01H 1/00; C12N 15/82

(52) U.S. Cl. 800/278; 435/468

(57) ABSTRACT

The invention concerns the application of short fragment homologous recombination (SFHR) to plant cells. In particular, the invention concerns the type of SFHR in which the short fragment is a single stranded nucleic acid preparation of one strand that is substantially free of the complementary strand.

### BACKGROUND OF THE INVENTION

The modification of the genome of a cell can, in principle, be accomplished either by introducing a complete gene into the genome at a random position or by making a specific alteration in an existing, naturally occurring gene. The former method when applied to plants results is plants that are variously termed genetically modified (GM) or transgenic and has been met with grave reservations by the general public, particularly by those with high levels of concern about environmental issues. To accommodate these reservations extensive testing has been required before approval. By contrast the plant that results from a specific targeted genetic alteration is indistinguishable from the plant that has been developed by a process of breeding and selection, the distinction being only that the process can be greatly accelerated because the genetic modifications can be directed rather than random.

In mammalian cells endogenous enzymes that effect homologous recombination have been used to introduce disruptions in specific genes for more than a decade. The technique is termed homolgous-recombination dependent gene targeting (hrdGT). Doetschman, T., et al., 1987, Nature 330, 576-78; Thomas K. R. & Capecchi, M. R., 1987, Cell 51, 503-12. These efforts involve the introduction of large pieces (several kilobases (kb)) of duplex DNA into the cell in the presence of a genetic selection system that distinguishes between homologous recombination and random insertion. Prior efforts towards the homologous recombination of large duplex DNA fragments in plants, although known, Kempin, S. A., et al., Nature 389, 802-3, have been less than uniformly successful. Reviewed: Oh & May, 2001, Curr. Op. Biotech. 12, 169-172; Hohn, B., & Puchta, H., 1999, PNAS 96, 8321-23; Mengiste, T., & Paszkowski, J., 1999, Biol. Chem. 380, 749-58.

The source of the difficulty is that the rate of homologous recombination in plant cells is lower compared to the rate of random insertion by illegitimate recombination than it is in animal cells. Thus in plant cells hrdGT has been proved practical.

Attempts to overcome the limitation by the expression of foreign genes in plant cells have been made. Reiss, B., et al., 1996 PNAS 93, 3094-98 (use of RecA); Shalev, G., et al., 1999. PNAS 96, 7398-02 (use of resolvase RuvC). These methods have had limited success in producing effect gene targeting. Reiss, B., et al., 2000, PNAS 97, 3358-63. Moreover, even when these modified cell are used to effect homologous recombination, the resultant modified cell would still contain an exogenous gene used to select the homologous recombinants and be considered a GM plant by regulators and the environmentally concerned public.

A method utilizing specific recombination sites and recombinases derived from transposons, rather than homologous recombination, has also been described in the patent literature. Baszczynski, C., et al., WO 01/85969; id, WO 99/25821.

An alternative method involves the use of self-complementary synthetic oligonucleotides that cause the specific alteration of one or two nucleotides. Such oligonucleotides typically contain a sequence of twenty to thirty base pairs, nearly all of which is homologous to a target gene, except for a single centrally located base pair that is not homologous, but rather designed to cause a site specific mutation. The oligonucleotides contain both deoxyribonucleotides and chemically modified RNase-resistant ribonucleotides. Reports in the scientific literature indicate that chimeric DNA/RNA duplex oligonucleotides can be effective to introduce targeted genetic modifications. Zhu, T., et al., 1999, PNAS 96, 8768-73; Beetham, P. R., et al., 1999, PNAS 96, 8774-78. More extensive descriptions are provided in the patent literature. Arntzen et al., WO 99/07865; Basczcynski et al., WO 99/25853.

Whether short duplex oligonucleotides cause genetic alteration by homologous recombination or by use of mismatch repair enzymes is not settled, but their mechanism is presumed to mismatch repair. The mixed structure of the oligonucleotide would likely prevent true recombination by genomic integration. Further evidence against recombination is provided by the observation that the genetic alteration occurs nearly as frequently one or two bases upstream of the predicted location as in the predicted location. Arntzen supra; Zhu, T., et al., 2000, Nature Biotech. 18, 555-58.

Efforts to develop cell-free systems to investigate and optimize oligonucleotide for the genetic alteration of plants have been reported. The systems use plant-cell extracts as a source of enzymes, chimeric oligonucleotides and plasmids containing bacterial antibiotic resistance markers to model the target gene. Readout is conveniently by counting bacterial transformants by modified plasmids. Gamper et al., 2000, Nucleic Acid Research 28, 4332-39; May, G., et al., WO 01/14531. However, to date there is no data indicating that the parameters that optimize the cell-free system would in fact optimize the modification of living plants cells. The potentially relevant source of differences include not only differences between a cell-free extract and the living, cellular system, but also differences between chromosomal DNA (i.e., histone-containing chromatin) and supercoiled bacterial plasmid DNA. Furthermore, extracts from cells lacking major mismatch repair enzymes appear to be fully active in the cell-extract assays, which suggests that some or all of the enzymes involved in the cell-free systems are bacterial. Id.

The use of a third alternative has been described in mammalian cells, but not plant cells. The technique is termed single-stranded short fragment homologoous replacement (ssSFHR). A DNA fragment of intermediate size, typically 400 to 800 bp is manufactured by excision from a plasmid vector or, alternatively, synthesized by PCR from a template. The short fragment is denatured by heat and the complementary strands can be optionally purified from each other. The technology is described in U.S. Pat. No. 6,010,908 by D. C. Gruenert, and in the scientific literature. Kapsa, R., et al., 2001, Human Gene Therapy 12, 629-42 (repair of murine dystrophin, unseparated strands); Colosima, A., et al., 2001, Mol. Therapy Vol. 3, No. 3 (episomal DNA in mammalian cells, unseparated strands); Goncz, K. K., et al., 1998, Hum. Mol. Genetics 7, 1913-19 (human cystic fibrosis transmembrane conductance regulator (CFTR), unseparated strands); Kunzelman, K., et al., 1996, Gene Therapy 3, 859-867 (murine CFTR, unseparated strands).

The ssSFHR technique differs from hrdGT in several respects. The nucleic acid is shorter (400-800 nt) compared to several kb for hrdGT; in ssSFHR the exogenous polynucleotide is denatured, i.e., single stranded, but is homologous with the target gene except for a few mutator nucleotides, in hrdGT foreign genes are embedded in the exogenous nucleic acid; and, in hrdGT a selection system is employed that distinguishes between homologous and illegitimate recombination, where in ssSFHR no such selection is required because illegitimate recombination does not occur at rates comparable to that of homologous recombination.

The prior art also includes the use of plasmid-carrying Agrobacterium tumefaciens bacteria for the construction of genetically modified plants. A part of the life cycle of the plasmid involves infection of plants. A. tumefaciens introduces the plasmid into the nucleus of plants in the form of a single strand. Yusibov, V. M., 1994, PNAS 91, 2994-98. A recombinant A. tumefaciens plasmid can be used to introduce exogenous DNA into a plant cell. The transferred part of the recombinant plasmid (the “T-DNA”) contains a 25 nt terminal sequence that is recognized by A. tumefaciens virulence protein and the entire single strand is coated by another virulence protein. The prior art includes a variant of this technique, in which the plasmids encoding the virulence proteins are co-transfected with the T-DNA sequence that is to be incorporated into the host plant genome. Such co-transfection results in the generation of the single strand DNA to be integrated in planta. Hansen, G., et al., 1997, PNAS 94, 11726-30; Hansen, G., & Chilton, M-D., 1996, PNAS 93, 14978-83. This technique is termed “agrolistic” transfection, because the technique uses both Agrobacterium-based technology with biolistic or accelerated particle technology.

### SUMMARY OF THE INVENTION

The invention provides a method of making a targeted genetic change in the genome of a plant cell. The method requires cloning of a target gene or a relevant fragment thereof, and introduction of a desired alteration into the cloned target gene by conventional in vitro site-directed mutagenesis. The mutated cloned gene is used as a template to generate a short fragment (henceforth “SF”) of between 200-1000 bp, preferably between 400 and 800 bp using conventional oligonucleotide primed polymerase chain reaction amplification. There can be more than one genetic alteration encoded in an SF, but the alterations should be limited in size and extent so that not more than four consecutive nucleotide of the SF will not be homologous to the target gene. The differences between the sequence of the SF and that of the target gne (The “heterologies”) can be either mismatches, insertions or deletions.

The SF is converted to single strand SF (“ssSF”), which can be used in either an unseparated complementary form (“ussSF”) or in a strand separated form (“s⁴SF”). The embodiment using s⁴SF may be employed to avoid reconversion of ussSF back to its double stranded form. The sequence of the SF will preferably be examined to determine self-complementary sequences that will cause extensive self-complementary secondary structure.

Once formed, the ssSF can be introduced into the plant cell by any of the methods that can be used to introduce duplex DNA into plant cells. The identification of modified cells and the generation of plants from those cells can then be performed according to conventional techniques well known in the field.

### DETAILED DESCRIPTION OF THE INVENTION

The invention provides for a method of making targeted genetic changes in the genome of plant cells and the culture of those cells into plants. The invention consists of the use of a short fragment (SF) of single stranded DNA of between 200 and 1000 nt and, more preferably between 400 and 800 nt. The single stranded SF can be provided in a mixture of complementary stands (“ussSF”), but strand-separated single strand SF (“s⁴SF”) are more stable and can be used. The sequence of the SF is designed to have the sequence that is desired to be introduced into the genome at the target gene.

Construction of the desired sequence can be most readily accomplished by in vitro site-directed mutagenesis. The techniques involved are well known in the art. Perrin, S., & Gilliland, G., 1990, Nucleic Acid Research 18, 7433; Landt, O., et al., 1990, Gene 96, 125-8; Nassal M., & Rieger, A., 1989, Nucleic Acids Research 18, 3077-8; Hemsley, A., et al., 1989, Nucleic Acids Research 17, 6545-51. Implementation of these techniques require that the target gene or a fragment of the gene that encompasses the sequenced to be modified is available in recombinant clones. Having constructed the appropriate desired sequence the SF itself can be synthesized by routine polymerase chain reaction (“PCR”). When s⁴SF are to be used, the synthesis employs one 5′-biotinylated primer and one underivitized primer. The strands are separated as described below. The synthesis of 5′-biotinylated primers is well known. Cook, A. F., et al., 1988, Nucleic Acids Research 16, 4077-95; Connolly, B. A., 1988, Nucleic Acids Research 15, 3131-9.

In one embodiment of the invention a targeted alteration can be made that leads to herbicide resistance. Targets and specific alterations include the alterations in the acetolactate synthase (ALS) gene (also termed the acetohydroxyacid synthase AHAS) that render the cell chorsulfuron(Glean™)-resistent. Chorsulfuron-resistant AHAS alterations are described in detail in WO 99/07865, WO 99/25853 and in Zhu et al., 2000, supra. In a second embodiment the target gene is 5-enolpyruvyl-3-phosphoshikimate synthase (“EPSPS”). Specific alterations in EPSPS that render a plant resistant to glyphosate (Roundup™) or paraquat are described in WO 98/54330 and WO 97/04103. The generation of EPSPS-modified plants is a particularly attractive use of the present invention because obtaining optimal results requires introduction of three alteration in a region of about 5 amino acids (173-178).

The invention is applicable to all commercially relevant crop species, to decorative plants and lawn grasses. Specific crop species include rice, maize, wheat, soy, canola, sesame, sun flower, cotton and tobacco.

In addition to the introduction of specific herbicide resistance into strains, an embodiment of the invention consists of the modification of plant cells by the specific inactivation (“knock-outs”) of certain genes whose expression causes undesired characteristics. Examples of such genes are found in WO 99/07865.

Yet a further use of the present invention is the silencing of genes of known sequence but unknown function. The resultant plants termed “knock-out” variants can be used to study the function of the gene.

After the SF is synthesized in a duplex form, i.e., the form in which the fragment is Watson-Crick bound to its complement, a single stranded SF can be prepared. The preparation is most simply accomplished by heat denaturation (heating to 95° C.) followed by rapid cooling to 4° C. This process results in a mixture of strands of both polarity having no or essentially no intermolecular Watson-Crick base pairings. However, continued incubation of the mixture at elevated temperatures can result in the formation of inter-molecular Watson-Crick pairings.

The separation of the complementary strands can be readily accomplished when one of the two primers used in the PCR synthesis of the SF is biotinylated. Separation of the product can be effected by binding the biotinylated strand to immobilized avidin as follows:
Double stranded SF (ds-SF) products can be prepared by PCR using two primers, one of which contained a biotin at the 5′ end.

Single Strand Preparation:
Single strands were generated by binding the biotinylated PCR product to avidin-magnetic beads (Dyonex).
The displaced strand (D-ssSF not containing biotin) was isolated by denaturing the bound dsPCR fragment under high pH (0.5 M NaOH) 1-2 minutes.
The “displaced strand” (supernatant) was removed from the beads using a magnet or centrifugation, neutralized with acid (27 ul cHCL per 500:1 0.5 M NaOH) and dialyzed (1000× volume 0.1 M Tris pH 7.0, then 2 Times 1000× volume of water).

The displaced strand was then concentrated by ethanol precipitation or spin concentrators.
The immobilized strand (B-SF) attached to the beads was neutralized with 2 Tris 0.1 M pH 7.0 washes followed by 2 water washes. The immobilized strand was removed from the magnetic beads in water following heat treatment (95° C.).

Both displaced and immobilized strands individually have activity. Typically the displaced strand was more active. Either the coding or non-coding strand may be used to introduce the modification into the targeted gene.

The s⁴SF or ussSF can be introduced into the plant cell by any method that can be used to introduce duplex DNA into plant cells for making transgeneic plants. For general review see Newell, C. A., 2000, Mol. Biotechnology 16, 53-65. Particular methods include: microinjection of protoplasts, Holm P. B., et al., 2000, Transgenic Research 9, 21-32, Schnorf, M., et al., 1991, Transgenic Res. 1, 23-30; protoplast electroporation, Bates, G. W., 1999, Methods Mol., Biol. 111,359-66, Jones, H., 1995, Methods Mol. Biol. 49, 107-12; pollen electroporation, Saunders, J. A., & Matthews, B. F., 1995, Methods Mol. Biol. 55, 81-88; biolistic particle bombardment, Finer J. J., 1999, Cur. Topics Microbiol Immunol. 240, 59-80, Daniell, H., 1997, Methods Mol. Biol. 62, 463-89, Ahl Goy, P. & Duesing, J. H., 1995, Bio/Technology 13, 454-458.

The amount of SF that need to be introduced into the plant cell is not critical. Guidance can be obtained from the amounts that have been used in making genetically modified plants by the above techniques and amounts that have been used in making genetic alterations using self-complementary synthetic oligonucleotides. For example, when biolistic delivery is used 0.5:g of s⁴SF can be precipitated onto 25:g of 1.0:gold particles using a precipitation reaction of 15:1 2.5 M CaCl₂ followed by 5:1 0.1M spermidine. See also WO 99/07865 at page 21 and Beetham, 1999. supra

The present invention should be distinguished from the Agrobacterium and Agrolistic technologies. Although in both of the above a single stranded DNA is introduced indirectly into a plant cell, the strand is integrated into the host cell genome and does not recombine with the host cell. Accordingly, in the prior art method the exogenous DNA needs to encode the entirety of a functional gene, while in the present invention the ssSF is homologous with a pre-existing target gene that is modified is situ.

The person skilled in the art will understand that the SF contains the sequence of the target gene including the desired modification. In contrast to Agrobacterium-related technology the SF need not include any sequences that are binding sites for Agrobacterium virulence proteins. In contrast to hrdGM techniques the SF need not contain selectable makers that are lost when homologous recombination occurs that can be used to distinguish homologous recombination from illegitimate recombination. The use of the term “having essentially the sequence of the targeted gene” herein is intended to exclude the above sequences when intended to be used in the method of the prior art, while permitting the SF to contain sequences unrelated to the sequence of the target gene as modified that are not essential to the function of the SF. As used herein the term “targeted gene” refers to so much of the target gene as is homologous with the SF and does not require that the SF encompass the entire target gene. The sequence of the SF need not be derived entirely from the sequence of an exon (coding region) of the target gene, intronic sequences can be used as well.

The modified target gene can be identified by any technique now known or to be developed. When the modified target gene gives rise to a herbicide resistant phenotype the modified target gene can be identified by selection with the herbicide. Other grossly observable markers in test systems include the activation of green fluorescent protein (Beetham. 1999, supra., Zhu, 1999, supra.)

Alternatively, the modified target gene can be identified by cloning and PCR testing of the cloned cells prior to the regeneration of whole plants. When identification of the modified gene by PCR is contemplated it is necessary to make alteration of a convenient restriction enzyme site in the genome, in addition to the desired mutation, so that the modified and unmodified PCR products can be readily distinguished by restriction enzyme digestion.

1. A method of making a genetic modification in a target gene of a plant cell comprising:
a) providing a DNA fragment having a length of between 400 and 800 nt and having essentially the sequence of the targeted gene as modified or of the complement thereof, which fragment is not Watson-Crick bound to another nucleic acid;
b) introducing the DNA fragment into the plant cell; and
c) identifying the presence of the modified target gene.

2. The method of claim 1, wherein the DNA fragment is made substantially free of a complementary DNA prior to its introduction into the plant cell.

3. The method of claim 2, which further comprises the generation of a plant from the plant cell.

4. The method of claim 2, wherein providing the DNA fragment comprises separating a biotinylated DNA strand from a complementary non-biotinylated DNA strand, wherein the fragment is either the biotinylated strand or the non-biotinylated strand.

5. The method of claim 1, which further comprises the generation of a plant from the plant cell.
